(19) United States
(12) Patent Application Publication
(10) Pub. No.: US 2018/0215792 A1
(43) Pub. Date: Aug. 2, 2018

(54) TWO-DIMENSIONAL STRUCTURES FROM PEPTOID OLIGOMERS AND METHODS OF MAKING

(71) Applicant: BATTELLE MEMORIAL INSTITUTE, Richland, WA (US)

(72) Inventor: Chunlong Chen, Richland, WA (US)

(73) Assignee: BATTELLE MEMORIAL INSTITUTE, Richland, WA (US)

(21) Appl. No.: 15/880,768

(22) Filed: Jan. 26, 2018

Related U.S. Application Data
(60) Provisional application No. 62/451,478, filed on Jan. 27, 2017.

(51) Int. Cl. C07K 14/00; A61K 47/69; C07K 7/06; A61K 49/00; A61K 31/704; B01D 71/58; B01D 61/02

(52) U.S. Cl. CPC C07K 14/001; A61K 47/6951; A61K 47/6953; C07K 7/06; B01D 61/027; A61K 49/0056; A61K 31/704; B01D 71/58; A61K 49/0043

(57) ABSTRACT

Materials and methods for forming self-assembled peptoid structures that are extremely stable, crystalline, free-standing and self-repairing are described. Based on the peptoid design, peptoid membranes in a 2D arrangement was able toroll into single-walled nanotubes with tunable sizes, diameters, thicknesses and stiffnesses as well as tailorable functions result. Crystalline nanomaterials made through this facile solution crystallization and anisotropic formation process are highly tailorable and exhibit a number of properties advantageous for applications such as water decontamination, cellular adhesion, imaging, surface coating, biosensing, energy conversion, biocatalysis or other applications.

### PRIORITY

This application claims priority from and incorporates by reference in its entirety U.S. Provisional Patent application No. 62/451,478 entitled Therapeutic Applications for Two-Dimensional Peptoid Oligomers filed 27 Jan. 2017.

### STATEMENT AS TO RIGHTS TO INVENTIONS MADE UNDER FEDERALLY-SPONSORED RESEARCH AND DEVELOPMENT

This invention was made with Government support under Contract DE-AC0576RL01830 awarded by the U.S. Department of Energy. The Government has certain rights in the invention.

### BACKGROUND OF THE INVENTION

In the field of materials the use and development two-dimensional (2D) structures such as graphene has received increasing interest in the design of new materials and devices, particularly on the micro and nano scale. In particular the use of such materials is of interest in applications such as bioanalytical devices, electrochemical devices (such as batteries, fuel cells and supercapacitors), membranes for filtration and separation, surface coatings with chemically defined surfaces and sensors to name a few. However, the widespread use of many known 2D materials is limited because the costs are high and the difficulties in engineering, producing and deploying the materials are significant. Furthermore, many of the attempts that have been made through molecular assembly have resulted in structures and materials that lack the robustness for effectual use or meaningful deployment. This is particularly true when the desired application includes interaction with complex conditions such as a therapeutics or drug delivery devices when harsh conditions such elevated temperatures, pressures, pH's, high salt or other conditions exist.

A desire therefore exists to obtain tailorable robust materials that form easily, have a desired ruggedness and provide simple solutions for meeting designated aims. Examples of such aim include but are not limited to mimicking a biological feature such as a cell membrane that performs molecular separations or for capturing or sensing a target material of interest from an environment or for displaying specific biological activity or for performing a task in a microelectronic environment. The present disclosure provides examples of significant advances in this area.

Additional advantages and novel features of the present invention will be set forth as follows and will be readily apparent from the descriptions and demonstrations set forth herein. Accordingly, the following descriptions of the present invention should be seen as illustrative of the invention and not as limiting in any way.

### SUMMARY

The present disclosure provides a new method to synthesize a novel class of nano materials from preselected peptoid oligomers in a liquid solution for a preselected period of time whereby the amorphous oligomers self-assemble to form crystalline 2D nanomaterials. Depending upon design and process conditions including the design of the peptoid oligomer, the processing conditions and the amount of time left in solution a variety of structures and shapes including membrane-mimetic two-dimensional (2-D) nanosheets, folded sheets, nanotubes and other structures. In some instances this self-assembly takes place via crystallization in solution while in other instances the self-assembly takes place on a substrate and forms a coating. The resulting crystalline material can be structured to have atomically flat hydrophobic and hydrophilic surfaces, is effective at cellular adhesion and is capable of self-repair.

In some instances the peptoid oligomers include a preselected item configured to perform a function such as a connect with a preselected target, link with another item, delivering a drug, capturing a designated material, tracing the passage of a material within a biological matrix, sensing the presence of a material or performing another desired activity. In one particular embodiment the peptoid is functionalized at the N-terminus, with a lysine-like group having a CO2 binding affinity for CO2 capture. The self-assembled structures described herein are extremely stable, crystalline, free-standing and self-repairing. Their related crystallization and anisotropic formation process provide a significant advance in the materials arena. In one embodiment these peptoid membranes exhibit a number of properties similar to those associated with cell membranes, including forming thicknesses in the 3.5-5.6 nm range, spontaneous assembly at interfaces, thickness variations in response to changes in Na+ concentrations, and the ability to self-repair. While the selection of the underlying peptoids with alternating hydrophilic and hydrophobic di block structures can be utilized to form lipid like membranes the described nanosheets are superior to lipid bilayers and other membrane-mimetic 2D nanomaterials assembled from lipid analogues because: they are highly stable, free-standing, and atomically ordered.

In addition they allow for the attachment of a broad range of functional objects at various locations in the peptoid sequence while leaving the basic membrane structure intact and they serve as a robust platform to incorporate and pattern functional objects through large side-chain diversity and/or co-crystallization approaches. These nanomaterials represent a significant step in the development of biomimetic membranes for applications in water purification, surface coatings, biosensing, energy conversion, water management (filtration, support, absorbance, and retention), biocatalysis drug delivery, or other applications such as antibacterial and antifouling applications among others.

The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

FIG. 1) shows the structures of one embodiment of a highly stable and crystalline peptoid nano-sheet nano-membrane with a tunable composition and structure.

FIGS. 2(a)-2(c) show examples of various membrane forming peptoid sequences that contain functional groups at various positions.

FIGS. 3(a)-3(m) show examples of various functionalized peptoid molecules that include dyes, drugs and receptor targeting ligands.

FIG. 4 shows the formation of a nanotube from a nanosheet structure through the rolling up and closure mechanism described in the present description.

FIG. 5 shows examples of functionalized nanotube structures containing various preselected functional molecules. Other functional molecules include but are not limited to: drug molecules, inorganic clusters, receptor-targeting ligands

### DETAILED DESCRIPTION OF THE INVENTION

The following description includes examples of various embodiments. It will be clear from this description that the invention described is not limited to these illustrated embodiments but that a variety of modifications and embodiments are also possible, contemplated and enabled by this disclosure. Therefore the following descriptions should be seen as illustrative and not limiting and including all modifications, alternative constructions, and equivalents falling within the spirit and scope of the invention as defined in the claims.

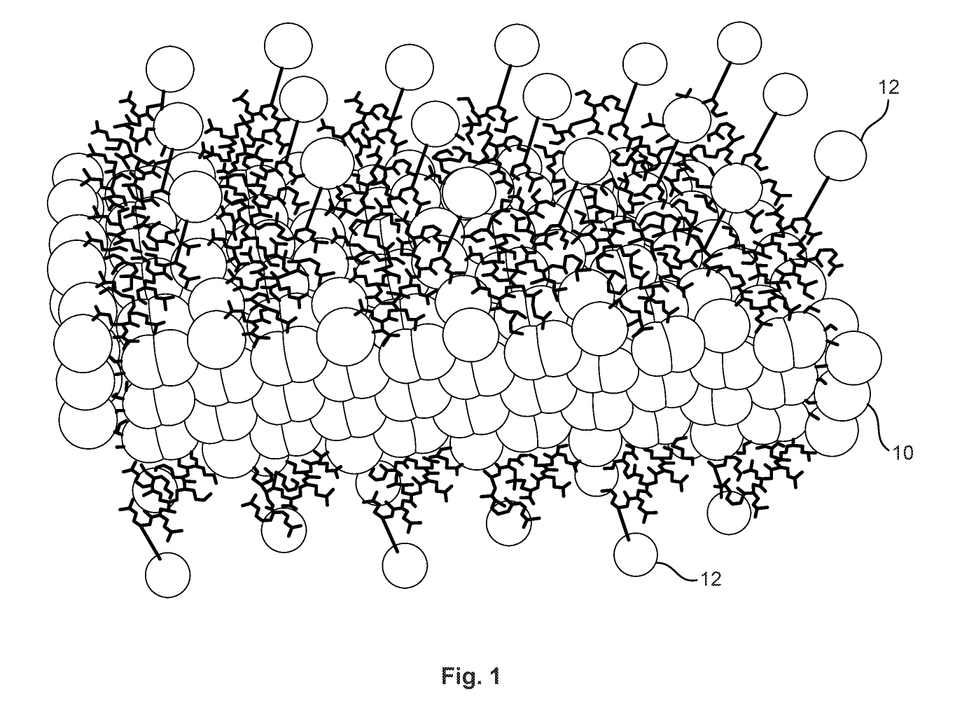

FIGS. 1-5 show various embodiments and descriptions of highly stable, membrane-like two dimensional sheet structures made from peptoid oligomers that can be variously tuned to create structures including free-standing nanomembranes, coatings, and even highly dynamic and stiff nanotubes. These materials can be configured, functionalized and/or loaded with any of a variety of various target materials so as to facilitate or perform a variety of desired but heretofore very difficult or impossible tasks. These highly-stable and free-standing nanomembranes exhibit self-repair and single-layer coating capabilities, and are formed through a spontaneous self-assembly crystallization process from solution. Their creation does not require interface assisted monolayer compression that attempts in the prior art have required nor are they susceptible to destabilization even when placed in organic solvents. An exemplary view of one such structure is shown in FIG. 1(a). FIG. 1 shows an assembly of peptoids wherein a portion of the peptoids are assembled to create a nanosheets structure 10 having a series of functionalized molecules 12 extending therefrom. In addition to these elements, additional functionalization or treatments can add other materials of interest or side chains to these structures.

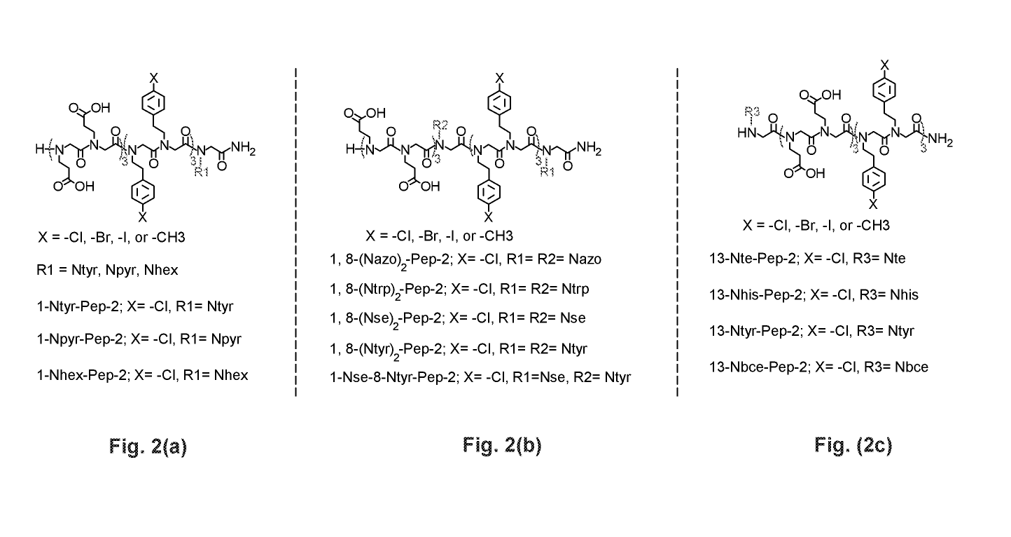

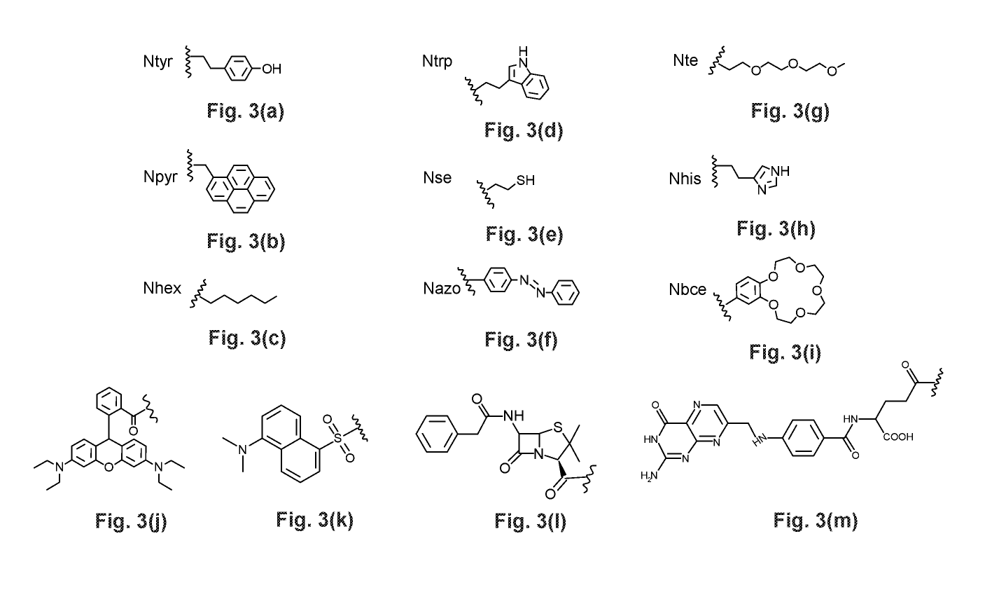

Peptoids, or poly-N-substituted glycines, are sequence-defined synthetic molecules that mimic both the structure and function of peptides and proteins, and bridge the gap between biopolymers and synthetic polymers. They are biocompatible and can be easily and cheaply synthesized. In contrast to peptides and proteins, peptoids are highly chemically and thermally stable and offer unique advantages for controlling assembly because lacking backbone hydrogen bonding allows the explicit introduction of interactions through the side chains, thereby leading to functions with high predictability. Examples of such molecules are shown in FIGS. 2(a)-2(c)). FIGS. 3(a)-3(m) shows examples of various functionalizations that could take place on these underlying peptoids including the addition of dyes, drugs, and receptor targeting ligands among others.

Using these peptoid oligomers as a base, a new class of highly stable and self-repairing 2D peptoid nanomembranes were created using a self-assembly process. The resulting nanomaterials are e free-standing and easy to functionalize. This crystallization method of formation provides the ability to pattern objects into assembled 2D nanomaterials and is significantly different from all previous peptoid 2D materials, which do not demonstrate co-crystallization and are therefore are highly challenged to tolerate the incorporation of bulky functional objects or to create nanoscale-patterning of functional objects within an assembly.

These membrane-mimetic 2D nanomaterial structures can be configured to incorporate and pattern a wide range of functional objects with long-range order to enable collective behaviors (e.g., enhanced structural stability, the ability to self-repair and the high photostability of fluorescent membranes). This represents a long sought after goal in chemistry, materials science, biology and bioengineering.

In one particular application, 12-meric peptoid oligomers capable of self-assembling into two-dimensional sheet structures both in solution and on substrates were created. These 12-mers were synthesized on a synthesizer using a process similar to the one described in U.S. Pat. No. 8,445,632, (The contents of which are incorporated by reference in their entirety) or manually synthesized using a new developed and easy-to-use method set forth below.

In one application manual synthesis of these 12-mer peptoids took place as Rink amide resin (0.09 mmol) was used to generate C-terminal amide peptoids. In the synthesis procedure, the Fmoc groups on the resin were deprotected by adding 2 mL of 20% (v/v) 4-methylpiperidine/N,N-dimethylformamide (DMF), agitating for 40 min, filtering, and washing with DMF. For all DMF washes, 1 mL DMF was added and then agitated for 1 min (repeated five times). An acylation reaction was then performed on the amino resin by the addition of 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF for 5 times. Nucleophilic displacement of the bromine with different primary amines occurred by the addition of 1.5 mL of 0.6 M primary amine monomerin N-methyl-2-pyrrolidone (NMP), followed by the agitation for 10 minutes at room temperature. The monomer solution were filtered from the resin, and washed with DMF for 5 times. The acylation and displacement steps were repeated until the designed peptoid was synthesized.

In other arrangements Pep-3 was synthesized by mixing the resulting rink amide resins (0.09 mmol) containing Pep-2 obtained from automated solid-phase synthesis with a DMF solution of Fmoc-6-aminohexanoic acid (1.5 mL, 0.9 mmol) and 0.50 mL of 50% (v/v) N,N-diisopropylcarbodiimide (DIC)/DMF. The mixture was agitated overnight at room temperature, filtered, and washed well with DMF. The terminal Fmoc group was deprotected by adding 2 mL of 20% (v/v) 4-methylpiperidine/DMF. The mixture was agitated for 40 min, filtered, and washed well with DMF.

CD-APO2 (CD=β-cyclodextrin, APO stands for amphiphilic peptoid oligomers) was synthesized by mixing the resulting rink amide resins (0.09 mmol) containing APO2 obtained from automated solid-phase synthesis with a DMF solution of bromoacetic acid (1.5 mL, 0.9 mmol) and 0.30 mL of 50% (v/v) N,N-diisopropylcarbodiimide (DIC)/DMF. The mixture was agitated for 10 minutes at room temperature, filtered and washed with DMF for 5 times. In the nucleophilic displacement step, 1.5 mL of 0.3 M CD-NH2 in DMF and K2CO3 (100 mg, 0.72 mmol) were added, followed by the agitation for 3 days at 40° C. The monomer solution were filtered from the resin, washed with deionized water for 5 times, and then washed well with DMF.

Peptoids containing N-[4-(2-phenyldiazenyl)phenyl]glycines] (Nazo) were synthesized by mixing 13-Nazo-Pep-2: Rink amide resins (0.09 mmol) containing Pep-2 with 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF. In the nucleophilic displacement step, a NMP solution of 4-Aminoazobenzene (1.5 mL, 0.9 mmol) and tetrabutylammonium iodide (TBAI, 100 mg, 0.27 mmol) was added into the above resins, followed by the agitation for 2 days at 40° C. The resulting resins were first washed with deionized water for 5 times and then washed with DMF for 5 times.

Peptoids containing N-[benzo-15-crown-5-ether]glycines (Nbce) were synthesized by combining 13-Nbce-Pep-2 resins wherein during the nucleophilic displacement step, a NMP solution 4′-Aminobenzo-15-crown 5-Ether (1.5 mL, 0.9 mmol) and tetrabutylammonium iodide (TBAI, 100 mg, 0.27 mmol) were used, followed by the agitation for 2 days at 40° C. The resulting resins were first washed with deionized water for 5 times and then washed with DMF for 5 times.

Peptoids containing [2-(4-imidazolyl)ethylamine]glycines (Nhis) were obtained by mixing 13-Nhis-Pep-2: rink amide resins (0.09 mmol) containing Pep-2 with 1.5 mL of 0.6 M chloroacetic acid in DMF, followed by adding 0.30 mL of 50% (v/v) N,N-diisopropylcarbodiimide (DIC)/DMF. The mixture was agitated for 10 minutes at room temperature, filtered and washed with DMF. In the nucleophilic displacement step, a NMP solution of histamine (1.5 mL, 0.9 mmol) was added into the above resins, followed by the agitation for one hour at 40° C. The resulting resins were washed well with DM.

After introducing Nhis in the peptoid, chloroacetic acid was used instead of bromoacetic acid for all subsequent steps of acylation in order to reduce side product formation as described previously. In the displacement step, primary amines substituted the chloride atom under the condition of agitation 1 hour at 40° C. Peptoids containing N-[2-(1H-indol-3-yl)ethyl]glycine (Ntrp) were formed by mixing 13-Ntrp-Pep-2: Rink amide resins (0.09 mmol) containing Pep-2 with 1.5 mL of 0.6 M chloroacetic acid in DMF, followed by adding 0.30 mL of 50% (v/v) N,N-diisopropylcarbodiimide (DIC)/DMF. The mixture was agitated for 10 minutes at room temperature, filtered and washed well with DMF. In the nucleophilic displacement step, a NMP solution of tryptamine (1.5 mL, 0.9 mmol) was added into the above resins, followed by the agitation for one hour at 40° C. The resulting resins were washed with DMF for 5 times.

After introducing Ntrp in the peptoid, chloroacetic acid was used instead of bromoacetic acid for all subsequent steps of acylation in order to reduce side product formation as described previously. In the displacement step, primary amines substituted the chloride atom under the condition of agitation 1 hour at 40° C. Peptoids containing N-[(1-pyrenemethyl)]glycines (Npyr) were synthesized by mixing 1-Npyr-Pep-2: Rink amide resins (0.09 mmol) containing Pep-2 with 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF for 5 times. In the nucleophilic displacement step, a 3.0 mL methanol solution of 1-Pyrenemethylaminehydrochloride (0.9 mmol) and N,N-Diisopropylethylamine (DIPEA) (0.9 mmol) was added into the above resins, followed by the agitation for 30 minutes at room temperature. The resulting resins were washed with DMF for 5 times.

NHS-Rhodamine-labeled Pep-3 was synthesized by a process wherein, 2 mL of DMF solution of NHS-Rhodamine (0.9 mmol) and 0.50 mL of 50% (v/v) N,N-diisopropylcarbodiimide (DIC)/DMF were added to a solution after the synthesis of Pep-3 and mixed with resins containing Pep-3, followed by the agitation for overnight at room temperature. The monomer solution were filtered from the resin, and washed with DMF for 5 times.

The resulting peptoid oligomers resulting from this process have di-block like sequences with hydrophilic or polar side-chains on one end and hydrophobic or apolar side-chains on another end. See FIGS. 2 and 3. When placed into solution under prescribed conditions and allowed to set for a period of time these oligomers self-assembled into 2D nanomaterial membranes generally reflecting the structure shown in FIG. 1 and having desired functionality based upon the configuration of the materials.

Such alternative arrangements provide 2D structures include examples for bioconjugation and/or specific molecular recognition. The evaporation-induced crystallization allows for 12-mer peptoids to be directly used for self-assembly after solid-phase synthesis without purification. In one application, an assembled 2D nanomaterial structure created by this process produced highly stable, sequence-specific and atomically flat connection with both hydrophilic and hydrophobic surfaces (e.g. mica, glass, graphite surfaces). When performed on a substrate, these membranes are capable of covering an entire surface including a well plate.

In other embodiments these materials can act as substrates or scaffolds for tissue engineering, binding specific bio-targets, or act as highly photostable nanoprobes for single particle tracking, as well as in therapeutic, drug delivery or diagnostic applications. For example, covalently bonding a lysine like group on to self-assembling peptoid oligomers allows for 2D peptoid nanosheet structures that have specific CO2 binding affinity have applications as molecular membranes for CO2 separations.

Compared to other coating techniques using chemical reagents (e.g. poly-lysine, polyethyleneimine, and collagen) or using plasmonic cleaning, these assembled 2D materials possess a dramatically higher activity and have low cost (Discussed in below) because the multipoint binding can be achieved to specific bio-targets by displaying these 2D sheets with complementary sequences.

In one arrangement, free-standing nanosheet membranes having a ˜4 nm in thickness, up to 5.0 um in width and length in gram scales were self-assembled starting from peptoids that contain a large number of chemically diverse side-chain residues. The use of 12-mer peptoid oligomers in this application, kept the peptoid synthesis cost low (˜$10 per 120 mg of 12-mers; for surface coating: the cost of coating can be as cheap as $0.5 per square meter by using these peptoids). Self-assembly of our membrane-mimetic 2D nanosheets took place not only in solution without requirement of compressing monolayers at water-air or water-oil interfaces, but also on different substrates, thus providing an enhanced and low-cost methodology for atom level coating (coating cost: $0.5 per square meter) and enabling a variety nano and microlevel interactions.

In one particular arrangement this self-assembly of membrane-mimetic 2D nanomaterials from these lipid-like peptoid oligomers took place as lyophilized and HPLC-grade peptoid oligomers were dissolved in a mixture of water and acetonitrile (v/v=1:1) to make 5.0 mM clear solution, this clear solution was then transferred to 4° C. refrigerator for slow evaporation. Suspensions or gel-like materials containing a large amount of crystalline membranes were formed after a few days. Using this arrangement biocompatible and photostable and bio-targeting 2D nanomembranes were assembled from sequence-defined peptoids. The resulting 2D nanomaterials were shown to be capable of regulating the process of membrane receptor internalization, and possess the ability to be rationally tuned for targeted cancer cell imaging and quantitatively tracking their intracellular pathways within live cells using a single-particle tracking technique.

Nanomembranes assembled from these diblock-like peptoids are highly stable and exhibited salt-induced thickness changes. To test the stability of peptoid membranes, they were exposed to a range of solvents as well as high temperature. We demonstrated that peptoid membranes survived when they were placed in a mixture of water and organic solvents, or even in pure organic solvents, such as CH3CN and EtOH, for over 6 hours. They were also stable in 10×PBS buffer (pH 7.4), 1.0 M Tris-HCl buffer (pH 7.4), and 1.5 M NaCl, or after heating to 60° C. in water overnight. Next, we investigated whether these membranes would exhibit salt-induced thickness changes, as is observed with lipid-bilayers. In situ AFM studies showed that when the peptoid membranes were exposed to NaCl solution or PBS buffers of increasing concentration, their thicknesses increased by about 30% from ˜4.2 nm to ˜5.4 nm.

High photostability and quantum yields of peptoid 2D nanomembranes arise from the crystallinity of these nanomaterials. By tuning the surface charge of the 2D nanomembranes using various peptoids such as those with various functional groups, we demonstrated the ability to control the lysosome escape of these materials within live cells during processes. This could assist in a variety of treatments and diagnostics for diseases including drug delivery and other interventions. These 2D nanomaterials can also be used for real-time monitoring of delivery of anticancer drugs with high drug loading rate.

In one specific example, dansyl dye molecules were attached to the membrane-forming peptoids, to create a highly photostable fluorescent nanomembranes that exhibited tunable surface chemistry and tunable surface charging for use in the targeted cancer cell imaging and tunable cellular internalization of pathways within live cells.

In another specific example, a series of membrane-forming peptoids were synthesized and modified to by attaching functional groups to the N-terminus of membrane-forming peptoids (Pep-DNS, Pep-FA (FA=folic acid)). These peptoid oligomers were then mixed and co-crystallized using the method described above to form nanomembranes with a tunable density of FA for cancer cell targeting and for tuning the intracellular delivery pathways and kinetics of peptoid membranes. The synthesized highly-photostable nanomembranes can be ultra-sonificated and filtered to form a structure that falls within a narrow size distribution and exhibits tunable surface charges in biological environments. In one specific example, the resulting nanoprobes have a monodisperse nanosheets structure with an average diameter of ˜70 nm.

An important factor for fluorescent probe used in single particle tracking (SPT) is good photostabibity. Peptoid membrane nanoprobes described above kept stable fluorescence properties and structures without aggregation in water even after three months. The high photostability of these peptoid membranes was also confirmed by comparing them with the commercial organic fluorescent dyes Cy5, inorganic quantum dots, and even DNS itself. These results demonstrate that the long-range ordering of surface dye molecules is critical for the synergistic effects of enhanced photostability. This high photostability was also confirmed by experiments of using peptoid membranes as nanoprobes for imaging in the living cells. The nanoprobes showed excellent good in-cell fluorescence stability compared with common cyanine dye (labeling Actin), which allowed us to use it as an endocytic probe in living imaging of cells.

Because peptoid membranes are biocompatible and exhibit high surface area, we expect that these highly stable peptoid membrane nanoprobes offer novel platforms for biological applications. To demonstrate the suitability advantage of nanoprobes as therapeutic agents besides their application as single particle tracking tag, we used nanoprobes as drug carriers to load doxorubicin (DOX)—a commercial drug that widely used in cancer chemotherapy because the aromatic domains of nanoprobes can assist the loading of DOX. The data indicated that there was strong fluorescence quenching between the nanoprobes and DOX. Based on this quenching effect, the DOX-releasing process was in vitro evaluated. We demonstrated that these highly photostable nanomembrane can serve for real-time monitoring of drug release and uptake within living cells due to the time-dependent recovering fluorescence signal of both the nanoprobes and DOX.

The methods and systems described present invention could find application in other areas including molecular separations, electronics, catalysis, optics, energy storage, and biomedicine. Various examples of these membranes, for example those which consist of 3.0-6.0 nm thick bilayers, are of particular interest because they represent a class of 2D materials that have rather unusual properties, such as sequence-specific water and ion transport and the ability to self-repair. Other embodiments of sequence-defined synthetic polymers that mimic lipid amphiphilicity can allow for self-assembly and self-repairing. 2D membrane mimetics exhibit protein-like molecular recognition would revolutionize the development of functional 2D nanomaterials including biomimetic membranes.

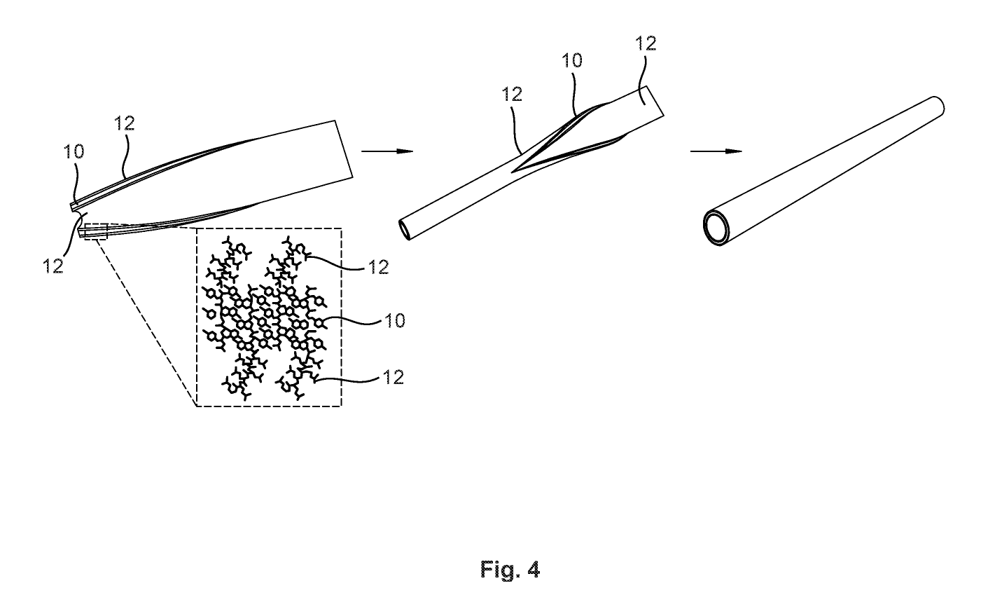

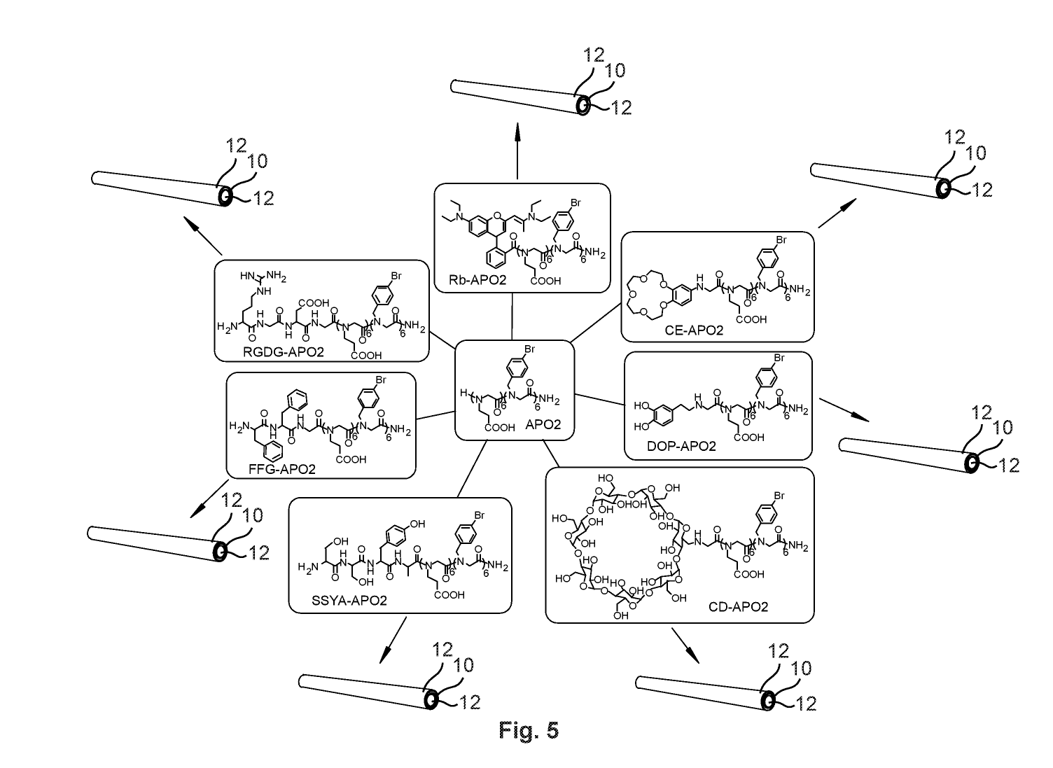

In addition to its arrangement as a tunable membrane, various preselected peptoid oligomers can self-assemble into a variety of shapes and configurations including a designable, stiff and dynamic single walled nanotube. Examples of which are shown in FIGS. 4 and 5. A shown in FIG. 4, formation of these materials takes place ins a process whereby the aforementioned crystalline nanosheet structures assembled from sequence-defined peptoids are rolled, folded and closed to obtain the desired result, single-walled peptoid nanotubes (SW-PNTs).

In one set of experiments peptoid oligomers were formed into PNTs in a process wherein peptoid solutions [5.0 mM, in water and acetonitrile (v/v=50:50, pH 2.5-3)] were left undisturbed at 4° C. for slow crystallization. Gel-like materials containing a large amount of crystalline free-floating PNTs were formed about two or three days later from amorphous phases. Testing on these materials revealed that uniform nanotubes exhibiting a wall thickness of 3.1±0.1 nm, similar to the thickness of bilayer-like peptoid membranes were formed with an average tube diameter of 37.2±2.7 nm. The tube height varied depending upon various conditions suggesting that peptoid nanotubes are dynamic enough to deform. These nanotubes exhibited a length over several micrometers and sonication proved to be an effective way to cut nanotubes in short sections. Interestingly, similar nanotubes formed even when the crystallization solution is at pH 7.4 or pH12. The fact that varying crystallization solution pH did not abolish nanotube formation suggests that hydrophobic interactions contribute significantly to stabilization and formation of nanotubes.

In one investigation, the nanotube formation process was slowed by reducing the concentration of peptoid oligomers to 0.5 mM to capture the nanotube intermediates. In this arrangement APO2 formed uniform nanospheres with a diameter of 26.2±5.1 nm after they were completely dissolved in the mixture of H₂O and CH₃CN. After slowly evaporating the solvent over 30 minutes at 4° C., TEM data showed that peptoids assembled into a mixture of nanospheres with a diameter of 44.9±7.5 nm and later into nanoribbon like sheets with a width of 75-120 nm and length of 200-600 nm. Between 1-72 hours of crystallization, the ribbons began to roll up, fold and close up to form elongated SW-PNTs.

These peptoid nanotubes provide a robust platform for developing biomimetic materials tailored to specific applications. Tuning their surface chemistry and the number of hydrophobic residues of peptoid oligomers allow for variation and tuning of the nanotube wall thickness, diameter and mechanical properties for a particular application. Varying the pH can trigger a reversible contraction-expansion motion of the nanotube. AFM-based mechanical measurements show PNTs can be assembled that are highly stiff (Young's Modulus ˜13-17 GPa).

Incorporation of preselected functional groups within PNTs allows for specialization for various deployments in a variety of applications. To demonstrate this, as shown in FIG. 3, we embedded a variety of chemistries into the structure: a fluorescent dye Rhodamine B (Rb-APO2), crown ether (CE-APO2), biomolecule dopamine (DOP-APO2), peptides sequences (FFG-APO2, SSYA-APO2, and RGDG-APO2), or cyclic host molecule β-cyclodextrin (CD-APO2) at the N-terminus of APO2. These items were made present as peptoid side chains, and the PNT assembly was sufficiently robust to tolerate the addition of functional groups, building functional PNTs with tunable compositions and functions. This ability provides a basis for the creation of a variety of types of structures suitable for performing a variety of tasks including but not limited to water decontamination, drug delivery, therapeutic, diagnostic and other nano and microscale applications.

In one example, such tunable PNTs were used for water decontamination. Dyes containing an azo chemical group (azo-dyes) have been widely used as textile colorants and now become one of the major toxic pollutants in water. Among various strategies that have been developed to remove azo-dyes, physical adsorption is considered to be superior to others due to its high efficiency, ease of operation and low cost. CD-APO2-PNTs were used here as adsorbents for the removal of azo-dyes from water, in which aromatic 4-aminoazobenzene was used as a model azo dye molecule. These CD-APO2-PNTs removed the majority of 4-aminoazobenzene molecules from water within one hour. We believe that this due at least in part to the combination of the function of β-cyclodextrin which is known to encapsulate azo-dyes, especially aromatics, through specific host-guest interactions and a nanotubular structure offers large surface area and high porosity.

In another set of experiments these PNTs were developed for promoting the cell adhesion. Specifically, the glass slide coated with RGDG-APO2-PNTs (FIG. 3) exhibited the most significant adhesion of A549 cancer cells in contrast to the control slides. Such promoted cell adhesion induced by RGD-containing PNTs was also observed during the uptake of sonication-cut-PNTs within A549 live cells.

The stability of these peptoid nanotubes was tested as we exposed them to a range of solvents as well as high temperature. Peptoid nanotubes survived when dispersed in alkaline solution (pH=11.94) for over 6 hours. The high stability of these peptoid nanotubes was further demonstrated as they remained intact after being incubated in a mixture of water and CH3CN, or in 1×PBS buffer, or in 1.0M NaCl, or after heating to 60° C. in aqueous solution for 3 hours. Peptoid nanotubes also survived when they were placed in pure organic solvents (e.g. CH₃CN and EtOH) for over 3 hours.

The present disclosure provides a variety of examples of new materials and materials for their synthesis. These materials in their various permutations allow for the specialization and tailoring of micro and nano structured devices that are useful in a variety of applications including but not limited to targeted material capture, for example as a part of environmental clean-up, or industrial processing or mining, or sensing or detection, in biological applications such as discovery, or diagnostics or therapeutic applications, in applications such as drug delivery, molecular sensing, biological imaging or biomimetic materials tailored to specific applications or application in nanoelectronics.

While various preferred embodiments of the invention are shown and described, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A method to synthesize a material comprising the step of:
placing preselected peptoid oligomers in a liquid solution for a preselected period of time whereby said oligomers self-assemble to form a crystalline material

2. The method of claim 1 wherein the crystalline material is a two dimensional nanosheet material.

3. The method of claim 2 wherein the self-assembly takes place on a substrate.

4. The method of claim 3 wherein the resulting crystalline material has atomically flat hydrophobic and hydrophilic surfaces.

5. The method of claim 2 wherein the self-assembly take place in solution and the crystalline material is free floating.

6. The method of claim 2 wherein the modified peptoid oligomers have alternating hydrophilic and hydrophobic side-chains.

7. The method of claim 6 wherein the modified peptoid oligomer is a 12-mer.

8. The method of claim 7 wherein the peptoid oligomers include a preselected complementary sequence configured to connect with a preselected target.

9. The method of claim 1 further wherein the solution further comprises a preselected material configured to perform a preselected function.

10. The method of claim 9 wherein the preselected function is delivering a drug.

11. The method of claim 9 wherein the preselected function is capturing a designated material.

12. The method of claim 9 wherein the preselected function is tracing the passage of a material within a biological matrix.

13. The method of claim 9 wherein the preselected function is sensing the presence of a material.

14. The method of claim 13 wherein the peptoid is functionalized at the N-terminus.

15. The method of claim 14 wherein the N-terminus comprises a lysine-like group having a CO2 binding affinity.

16. The method of claim 1 wherein crystalline material is a partially folded nanosheet.

17. The method of claim 1 wherein crystalline materials is a nanotube.

18. A two dimensional nanomembrane-like material comprising:
modified peptoid oligomers self-assembled in a crystalline structure.

19. The material of claim 18 crystalline material has atomically flat hydrophobic and hydrophilic surfaces.

20. The material of claim 18 wherein the peptoid oligomers include a preselected complementary sequence configured to connect with a preselected target.

21. The material of claim 18 wherein the crystalline structure further comprises a preselected material configured to perform a preselected function.

22. The material of claim 18 wherein the preselected function is delivering a drug.

23. The material claim 18 wherein the preselected function is capturing a designated material.

24. The material of claim 18 wherein the preselected function is tracing the passage of a material within a biological matrix.

25. The material of claim 18 wherein the preselected function is sensing the presence of a material.

26. The material of claim 18 wherein the peptoid is functionalized at the N-terminus to include a lysine-like group having a CO2 binding affinity.
